(19) United States
(12) Patent Application Publication
(10) Pub. No.: US 2002/0192215 A1
(43) Pub. Date: Dec. 19, 2002

(54) Novel uses of mammalian OX2 protein and related reagents

(75) Inventors: Robert M. Hoek, Mountain View, CA (US); Jonathan D. Sedgwick, Palo Alto, CA (US)

Correspondence Address: DNAX Research, Inc., 901 California Avenue, Palo Alto, CA, 94304-1104, US

(73) Assignee: Schering Corporation, a New Jersey corporation

(21) Appl. No.: 10/086,972

(22) Filed: Mar. 1, 2002

Related U.S. Application Data
(62) Division of application No. 09/547,432, filed on Apr. 12, 2000, now abandoned.
(60) Provisional application No. 60/129,124, filed on Apr. 13, 1999.

(51) Int. Cl. A61K 39/395; A61K 38/17

(52) U.S. Cl. 424/144.1; 514/12

(57) ABSTRACT

Compositions and methods for using mammalian ligand OX2 to treat an abnormal physiological condition in an individual. The methods comprise administering a therapeutically effective amount of OX2 alone, or in combination with other therapeutic reagents; or an OX2 antagonist.

The present application is a conversion to a U.S. Utility patent application of U.S. Provisional Patent Application U.S. S No. 60/129,124, filed Mar. 13, 1999, which is incorporated herein by reference.

### FIELD OF THE INVENTION

The present invention relates to methods of using proteins which function in controlling physiology, development, and differentiation of mammalian cells, e.g., cells of a mammalian immune or neural system. In particular, it provides methods of using proteins and mimetics which regulate cellular physiology, development, differentiation, or function of various cell types, including hematopoietic or neural cells.

### BACKGROUND OF THE INVENTION

The immune system of vertebrates consists of a number of organs and several different cell types. Two major cell types include the myeloid and lymphoid lineages. Among the lymphoid cell lineage are B cells, which were originally characterized as differentiating in fetal liver or adult bone marrow, and T cells, which were originally characterized as differentiating in the thymus. Another cell type is the mononuclear phagocyte, a cell lineage widely distributed throughout most tissues. The phagocytes play a role in inflammation, host defenses, and reaction against a range of autologous and foreign materials. See, e.g., Paul (ed. 1997) Fundamental Immunology (4th ed.) Raven Press, New York.

In many aspects of the development or regulation of an immune response or cellular differentiation, soluble or membrane proteins play a critical role in regulating cellular interactions. These proteins also mediate cellular activities in many ways. They have been shown, in many cases, to modulate proliferation, growth, and differentiation of hematopoietic stem cells into the vast number of progenitors composing the lineages responsible for an immune response. Others are important mediators of intercellular signaling, often as receptors or ligands. They are also quite important in immunological responses and physiology.

However, the cellular molecules which are expressed by different developmental stages of cells in these maturation pathways are still incompletely identified. Moreover, the roles and mechanisms of action of signaling molecules which induce, sustain, or modulate the various physiological, developmental, or proliferative states of these cells is poorly understood. Clearly, the immune system and its response to various stresses has relevance to medicine, e.g., clearance of cellular or other materials after injury, infectious diseases, cancer related responses and treatment, and allergic and transplantation rejection responses. See, e.g., Thorn, et al. Harrison's Principles of Internal Medicine McGraw/Hill, New York; Ziegler, et al. (ed. 1997) Growth Factors and Wound Healing: Basic Science and Potential Clinical Applications Springer Verlag; Clark (ed. 1996) The Molecular and Cellular Biology of Wound Repair Plenum; and Peacock (1984) Wound Repair Saunders.

Medical science relies, in large degree, to appropriate recruitment or suppression of the immune system in effecting cures for insufficient or improper physiological responses to environmental factors. However, the lack of understanding of how the immune system is regulated or differentiates has blocked the ability to advantageously modulate the immunological mechanisms to biological challenges, i.e., response to biological injury. Medical conditions characterized by abnormal or inappropriate regulation of the development or physiology of relevant cells thus remain unmanageable. The discovery and characterization of specific regulatory pathways and their physiological effects will contribute to the development of therapies for a broad range of degenerative or other conditions which affect the biological system, immune cells, as well as other cell types. The present invention provides solutions to some of these and many other problems.

### SUMMARY OF THE INVENTION

The present invention is based, in part, upon the discovery of the physiological role of the ligand OX2, also referred herein as the OX2 protein, in various models of immune response. In particular, the role of ligand OX2 has been elucidated in pathways involved in infectious disease, hematopoietic development, and viral infection.

The present invention provides methods of modulating the trafficking or activation of a leukocyte in an animal, the methods comprising contacting myeloid lineage cells, e.g., monocyte/macrophage, in the animal with a therapeutic amount of an agonist of a mammalian OX2 protein; or an antagonist of a mammalian OX2 protein. Preferred embodiments include where: the mammalian OX2 protein is a primate protein; and/or the antagonist is an antibody which binds to the mammalian OX2. Certain embodiments include where the myeloid lineage cells, e.g., monocyte/macrophage, include a macrophage, microglial, granulocyte, or a dendritic cell, or where the animal exhibits signs or symptoms of an infectious, inflammatory, leukoproliferative, neurodegenerative, or post-traumatic condition. Preferred embodiments include where the sign or symptom is in neural tissue; lymphoid tissue; myeloid tissue; pancreas; gastrointestinal tissue; thyroid tissue; muscle tissue; or skin or collagenous tissue.

Other methods include where the modulating is inhibiting function of the leukocyte cell; and/or where the administering is the agonist. Preferably, the agonist is the mammalian OX2. Certain embodiments include where the animal is experiencing signs or symptoms of autoimmunity; an inflammatory condition; tissue specific autoimmunity; degenerative autoimmunity; rheumatoid arthritis; atherosclerosis; multiple sclerosis; vasculitides; delayed hypersensitivities; skin grafting; a transplant; spinal injury; stroke; neurodegeneration; or ischemia. The administering may be in combination with: an anti-inflammatory cytokine agonist or antagonist; an analgesic; an anti-inflammatory agent; or a steroid.

Various other methods are provided where the modulating is enhancing function of the leukocyte cell, and/or the administering is the antagonist. Preferably, the antagonist is: an antibody which binds to the mammalian OX2; or a mutein of the mammalian OX2 which competes with the mammalian OX2 in binding to an OX2 receptor, but does not substantially signal. In various embodiments, the method is applied where the animal experiences signs or symptoms of infection, wound healing, or clot formation. The administering will often be in combination with: an angiogenic factor; a growth factor, including FGF or PDGF; an antibiotic or antiviral reagent; or a clotting factor.

Different methods are provided, e.g., of modulating the activation of a leukocyte in a tissue, the method comprising contacting myeloid or monocyte/macrophage lineage cells in the tissue with: an agonist of a mammalian OX2 protein; or an antagonist of a mammalian OX2 protein. Often the modulating is inhibiting the leukocyte cell, and the contacting is with the agonist. The administering is often in combination with: an anti-inflammatory cytokine agonist or antagonist; an analgesic; an anti-inflammatory agent; or a steroid. Alternatively, the modulating is enhancing, and the contacting is with the antagonist. The administering may be in combination with: an angiogenic factor; a growth factor, including FGF or PDGF; an antibiotic or antiviral; or a clotting factor.

### DETAILED DESCRIPTION OF THE PREFERRED EMBODIMENTS OUTLINE

I. General

II. Nucleic Acids

A. natural isolates; methods

B. synthetic genes

C. methods to isolate

III. Purified ligand OX2 protein

A. physical properties

B. biological properties

IV. Making ligand OX2 protein; Mimetics

A. recombinant methods

B. synthetic methods

C. natural purification

V. Physical Variants

A. sequence variants, fragments

B. post-translational variants

1. glycosylation

2. others

VI. Functional Variants

A. analogs; fragments

1. agonists

2. antagonists

B. mimetics

1. protein

2. chemicals

C. species variants

VII. Antibodies

A. polyclonal

B. monoclonal

C. fragments, binding compositions

VIII. Uses

A. diagnostic

B. therapeutic

IX. Kits

A. nucleic acid reagents

B. protein reagents

C. antibody reagents

I. General

The OX2 antigen was first characterized in rat, using a monoclonal antibody (mAb) MRC OX2. See, e.g., McMaster and Williams (1979) Eur. J. Immunol. 9:426-433; Barclay (1981) Immunology 44:727-736; Barclay (1981) Immunology 42:593-600; Bukovsky, et al. (1984) Immunology 52:631-640; and Webb and Barclay (1984) J. Neurochem. 43:1061-1067. Using this antibody in immunohistochemical (IHC) staining of tissue sections or cell suspensions for flow cytometry revealed that the OX2 antigen was expressed by a wide variety of cells, e.g., neurons, vascular endothelium, B cells, activated T cells, follicular dendritic cells, interdigitating dendritic cells, smooth muscle cells, and trophoblasts. Furthermore, human OX2 is known to be expressed in normal brain and by B cells. McCaughan, et al. (1987) Immunogenetics 25:329-335. Characterization of the rat protein recognized by MRC OX2 (Clark, et al. (1985) EMBO J. 4:113-118) revealed that OX2 consists of about 248 amino acids comprising two extracellular immunoglobulin (Ig) domains, a transmembrane domain and a short C-terminal cytoplasmic tail. The molecule is glycosylated through 6 N-linked glycosylation sites, three of which are present in the N-terminal V-like Ig. domain and the others reside in the membrane proximal C2-like Ig domain. This places OX2 in the Ig superfamily (IgSF), forming a sub-group of small IgSF molecules with molecules like CD2, CD48, CD58, CD80, CD86, CD90, and CD147. Interestingly, CD90 is also highly expressed by neurons. Williams, et al. (1977) Cold Spring Harb. Symp. Quant. Biol. 41 Pt 1:51-61. Furthermore, it was shown that OX2 was a structural homologue of CD80 and CD86 (Borriello, et al. (1997) J. Immunol. 158:4548-4554) and that the OX2 gene was closely linked to those coding for CD80 and CD86 on chromosome 16 in the mouse. Borriello, et al. (1998) Mamm. Genome 9:114-118. Both CD80 and CD86 serve as ligands in a process known as co-stimulation, and therefore it is likely that OX2 would act as a ligand as well. The OX2 antigen will be referred hereafter as the OX2 protein or ligand OX2. The binding partner will be referred to as the OX2 receptor, though it has not been fully characterized.

To identify the receptor for OX2 (OX2R) the group of Barclay prepared a multivalent reagent using rat OX2-rat CD4 fusion protein bound to fluorescent beads. This reagent was shown to bind to mouse and rat peritoneal macrophages, and this binding could be blocked by the mAb MRC OX88. Preston, et al. (1997) Eur. J. Immunol. 27:1911-1918. This mAb was shown to bind to macrophages isolated from both peritoneum and spleen and in IHC on spleen sections staining was found in areas known to contain high proportions of macrophages.

Defective or exaggerated activation of macrophages contributes to pathogenesis of a wide range of immunological and other diseases. See, e.g., McGee, et al. (eds. 1992) Oxford Textbook of Pathology Oxford University Press, Oxford; Lewis and McGee (eds. 1992) The Macrophage IRL Press, Oxford; and Bock and Goode (eds. 1997) The Molecular Basis of Cellular Defence Mechanisms Wiley & Sons.

The distribution of the OX2 is consistent with a hypothesis that OX2 relays a signal through the OX2R to macrophages, and possibly other cells of the myeloid or monocyte-macrophage lineages. In this scenario, for instance, expression of OX2 on neurons could establish a direct way of communication to the resident macrophages of the brain called microglia that might express OX2R, since they originate from the monocyte-macrophage lineage. Perry and Gordon (1988) Trends Neurosci. 11:273-277. Using the MRC OX88 mAb in IHC of brain sections it has not been possible to identify the molecule on microglia. However, this negative result could be caused by the fact that MRC OX88 is an IgM, an antibody isotype generally known to have low affinity.

To study the biological role of OX2, and in particular whether OX2-OX2R interactions are involved in regulation of macrophage function, a mouse OX2 genomic clone was isolated from a C57BL/6 genomic library. This allowed the construction of a targeting vector, with which knockout (KO) mice were created by targeted disruption of the OX2 gene by homologous recombination in C57BL/6 ES cells. The homozygous KO mice bred and developed normally, although initial examination of the internal organs showed anatomical anomalies in some lymphoid tissue. These included enlarged red pulp of the spleen, and failed segregation of the mesenteric lymph nodes with enlarged marginal sinus. Both these changes are attributable to an expanded macrophage and, in the spleen at least, an expanded granulocyte population. These results indicate that even in the steady state, OX2 may regulate myeloid cell, e.g., macrophage, numbers and their activation, presumably via ligation of OX2R.

The OX2 KO mice can now be used in studies of myeloid cell or macrophage function, particularly of monocyte/macrophage lineage activities, by applying model systems for activation of cells of these cell lineages. The first model system used for this purpose is a paradigm for microglia activation in the brain through nerve injury. Streit and Graeber (1993) Glia 7:68-74. This model makes use of the fact that transection of the facial nerve, that directs motor behavior in the facial area, elicits microglia activation after four to seven days in the facial nucleus in the brainstem, where the motor neurons are located. In the OX2 KO mice, this activation occurs already 2 days after surgery, much earlier than in a normal mouse. This activation is accompanied by expression of the activation marker DAP12, as shown by IHC.

Both the results of the steady state and the facial nerve transection are consistent with a hypothesis that ligation of the OX2R on macrophages by OX2 gives rise to a down-regulatory signal. This hypothesis can be studied in more detail and in different model systems, such as in vivo activation of cells of the monocyte-macrophage lineage, e.g., by intraperitoneal injections with LPS and determination of serum levels of TNF. In the OX2 KO mice the TNF response upon LPS challenge may be more robust, and the macrophages in these mice lack a particular down-regulatory mechanism.

If this hypothesized role of the OX2-OX2R interaction holds true, manipulation of this interaction can have important clinical implications. In settings where macrophage activation is desired, e.g., wound healing, some aspects of healing in CNS injury, etc., blocking of OX2 or using an OX2R antagonist would be beneficial. Release from the typical suppression will result in quicker or more pronounced activation. Enhanced granulocyte activity would also be beneficial for control of bacterial infection.

Conversely, in situations where macrophage activation should be suppressed, e.g., inflammation such as seen in rheumatoid arthritis, activation of the OX2R by agonists, e.g., a recombinant soluble OX2 in a multivalent form that can cross-link the OX2R, could be useful. This would delay or prevent release from active suppression.

The descriptions below are directed, for exemplary purposes, to primate, e.g., a human, or rodent, e.g., mouse or rat ligand OX2, but are likewise applicable to related embodiments from other species. Thus, conditions known to be mediated by or related to macrophage functions may be regulatable using these reagents.

II. Nucleic Acids

General description of nucleic acids, their manipulation, and their uses (including, e.g., complementary and antisense nucleic acids) are provided in the following references: NCBI Entrez Accession numbers (search for “MRC OX-2”) X05323-26 (human); X01785 (rat); AA924563, AF029214-216, and AH006102 (mouse); McCaughan, et al. (1987) Immunogenetics 25:329-335; Goodnow (1992) “Transgenic Animals” in Roitt (ed.) Encyclopedia of Immunology Academic Press, San Diego, pp. 1502-1504; Travis (1992) Science 256:1392-1394; Kuhn, et al. (1991) Science 254:707-710; Capecchi (1989) Science 244:1288; Robertson (ed. 1987) Teratocarcinomas and Embryonic Stem Cells: A Practical Approach IRL Press, Oxford; Rosenberg (1992) J. Clinical Oncology 10:180-199; Cournoyer and Caskey (1993) Ann. Rev. Immunol. 11:297-329; Wetmur and Davidson (1968) J. Mol. Biol. 31:349-370; Weintraub (1990) Scientific American 262:40-46; Jaroszewski and Cohen (1991) Advanced Drug Delivery Reviews 6:235-250; Akhtar, et al. (1992) pages 133-145 in Erickson and Izant (eds.) Gene Regulation: Biology of Antisense RNA and DNA Raven Press, New York; Zhao, et al. (1994) Blood 84:3660-3666; Misquitta, et al. (1999) Proc. Nat'l Acad. Sci. USA 96:1451-1456; and Treco WO96/29411, each of which is incorporated by reference. Additional aspects will be apparent to a person having ordinary skill in the art in light of the teachings provided herein.

III. Purified Ligand OX2 Protein

General descriptions of proteins and polypeptides in pharmaceutical or biochemical contexts can be found, e.g., in: Goodman, et al. (eds. 1990) Goodman & Gilman's: The Pharmacological Bases of Therapeutics (8th ed.) Pergamon Press; Avis, et al. (eds. 1993) Pharmaceutical Dosage Forms: Parenteral Medications Dekker, New York; Lieberman, et al. (eds. 1990) Pharmaceutical Dosage Forms: Tablets Dekker, New York; Lieberman, et al. (eds. 1990) Pharmaceutical Dosage Forms: Disperse Systems Dekker, New York; Freifelder (1982) Physical Biochemistry (2d ed.) W. H. Freeman; Cantor and Schimmel (1980) Biophysical Chemistry, parts 1-3, W. H. Freeman & Co., San Francisco. Specific descriptions of OX2 are found, e.g., in WO97/21450 (human); NCBI Entrez accession numbers (search MRC OX-2) include P41217 (human); P04218 (rat); and AAC15911 (mouse). Recombinant methods for making the protein are well known. Preparation of fragments by synthetic methods, or by biochemical cleavage of natural or recombinant forms, are available.

IV. Making OX2 Protein; Mimetics

DNA which encodes the ligand OX2 protein or fragments thereof can be obtained by chemical synthesis, screening cDNA libraries, or by screening genomic libraries prepared from a wide variety of cell lines or tissue samples.

This DNA can be expressed in a wide variety of expression systems as described in, e.g., U.S. Ser. No. 08/250,846; U.S. Ser. No. 08/177,747; U.S. Ser. No. 08/077,203; PCT/US95/00001; Kaufman, et al. (1985) Molec. and Cell. Biol. 5:1750-1759; Pouwels, et al. (1985 and Supplements) Cloning Vectors: A Laboratory Manual, Elsevier, N.Y., Rodriguez, et al. (eds. 1988) Vectors: A Survey of Molecular Cloning Vectors and Their Uses, Buttersworth, Boston, Mass.; Rodriguez and Denhardt (eds.) Vectors: A Survey of Molecular Cloning Vectors and Their Uses Buttersworth, Boston, Chapter 10, pp. 205-236; Okayama, et al. (1985) Mol. Cell Biol. 5:1136-1142; pMClneo Poly-A, see Thomas, et al. (1987) Cell 51:503-512; O'Reilly, et al. (1992) Baculovirus Expression Vectors: A Laboratory Manual Freeman and Co., CRC Press, Boca Raton, Fla.; Low (1989) Biochim. Biophys. Acta 988:427-454; Tse, et al. (1985) Science 230:1003-1008; and Brunner, et al. (1991) J. Cell Biol. 114:1275-1283; each of which is incorporated herein by reference.

Now that the various ligand OX2 proteins have been characterized, fusion polypeptides, fragments, or derivatives thereof can be prepared by conventional processes for synthesizing peptides. These include processes such as are described in Stewart and Young (1984) Solid Phase Peptide Synthesis Pierce Chemical Co., Rockford, Ill.; Bodanszky and Bodanszky (1984) The Practice of Peptide Synthesis Springer-Verlag, New York; Bodanszky (1984) The Principles of Peptide Synthesis Springer-Verlag, New York; and Merrifield, et al. (1963) in J. Am. Chem. Soc. 85:2149-2156; each of which is incorporated herein by reference. Additional aspects will be apparent to a person having ordinary skill in the art in light of the teachings provided herein.

V. Physical Variants

Proteins or peptides having substantial amino acid sequence homology with the amino acid sequence of the OX2 protein are also contemplated. The variants include species or allelic variants. Homology, or sequence identity, is defined in, e.g., U.S. Ser. No. 08/250,846; U.S. Ser. No. 08/177,747; U.S. Ser. No. 08/077,203; PCT/US95/00001; Needleham, et al. (1970) J. Mol. Biol. 48:443-453; Sankoff, et al. (1983) Chapter One in Time Warps, String Edits, and Macromolecules: The Theory and Practice of Sequence Comparison Addison-Wesley, Reading, Mass.; software packages from NCBI, NIH; and the University of Wisconsin Genetics Computer Group, Madison, Wis.

The isolated DNA encoding an OX2 protein can be readily modified as described in, e.g., Sambrook, et al. (1989); Ausubel, et al. (1987 and Supplements); Cunningham, et al. (1989) Science 243:1330-1336; O'Dowd, et al. (1988) J. Biol. Chem. 263:15985-15992; and Carruthers (1981) Tetra. Letts. 22:1859-1862; each of which is incorporated herein by reference. Additional methods will be apparent to a person having ordinary skill in the art in light of the teachings provided herein.

VI. Functional Variants

The blocking of physiological response to ligand OX2 proteins may result from the inhibition of binding of the ligand to its natural binding partner by a variant of natural OX2 or antibody to OX2. Methods for making such a variant are described in, e.g., Godowski, et al. (1988) Science 241:812-816; Beaucage and Carruthers (1981) Tetra. Letts. 22:1859-1862; Sambrook, et al. (1989) Molecular Cloning: A Laboratory Manual (2d ed.) Vols. 1-3, Cold Spring Harbor Laboratory; Merrifield (1963) J. Amer. Chem. Soc. 85:2149-2156; Merrifield (1986) Science 232: 341-347; Atherton, et al. (1989) Solid Phase Peptide Synthesis: A Practical Approach, IRL Press, Oxford; Cunningham, et al. (1989) Science 243:1339-1336; O'Dowd, et al. (1988) J. Biol. Chem. 263:15985-15992; and Lechleiter, et al. (1990) EMBO J. 9:4381-4390; each of which is incorporated herein by reference. Additional methods will be apparent to a person having ordinary skill in the art in light of the teachings provided herein.

VII. Antibodies

Antibodies can be raised to the various ligand OX2 proteins, including species or allelic variants, and fragments thereof, both in their naturally occurring forms and in their recombinant forms. Additionally, antibodies can be raised to ligand OX2 proteins in either their active forms or in inactive forms. Anti-idiotypic antibodies are also contemplated. Methods for generating antibodies and binding compositions and their uses are described in, e.g., Coligan (1991) Current Protocols in Immunology Wiley/Greene; Harlow and Lane (1989) Antibodies: A Laboratory Manual Cold Spring Harbor Press; Chan (ed. 1987) Immunoassay: A Practical Guide Academic Press, Orlando, Fla.; Ngo (ed. 1988) Nonisotopic Immunoassay Plenum Press, NY; Price and Newman (eds. 1991) Principles and Practice of Immunoassay Stockton Press, NY; (1969) Microbiology Hoeber Medical Division, Harper and Row; Landsteiner (1962) Specificity of Serological Reactions Dover Publications, New York; Williams, et al. (1967) Methods in Immunology and Immunochemistry, Vol. 1, Academic Press, New York; Stites, et al. (eds.) Basic and Clinical Immunology (4th ed.) Lange Medical Publications, Los Altos, Calif., and references cited therein; Harlow and Lane (1988) Antibodies: A Laboratory Manual CSH Press; Goding (1986) Monoclonal Antibodies: Principles and Practice (2d ed.) Academic Press, New York; Kohler and Milstein (1975) Nature 256:495-497; Huse, et al. (1989) “Generation of a Large Combinatorial Library of the Immunoglobulin Repertoire in Phage Lambda” Science 246:1275-1281; Ward, et al. (1989) Nature 341:544-546; U.S. Pat. Nos. 3,817,837; 3,850,752; 3,939,350; 3,996,345; 4,277,437; 4,275,149; and 4,366,241; and Cabilly, U.S. Pat. No. 4,816,567; each of which is incorporated herein by reference. Additional methods will be apparent to a person having ordinary skill in the art in light of the teachings provided herein.

VIII. Uses

Mammalian OX2 reagents will have a variety of therapeutic uses for, e.g., the treatment of conditions or diseases in which myeloid or macrophage cell function or dysfunction has been implicated. These would include, e.g., wound healing, some aspects of healing in CNS injury, and inflammation such as seen in rheumatoid arthritis. Administration of an effective amount of ligand OX2 will typically be at least about 100 ng per kg of body weight; usually at least about 1 ug per kg of body weight; and often less than about 1 mg per kg of body weight; or preferably less than about 10 mg per kg of body weight. An effective amount will modulate the symptoms, or time to onset of symptom, typically by at least about 10%; usually by at least about 20%; preferably at least about 30%; or more preferably at least about 50%. The present invention provides reagents which will find use in additional diagnostic and therapeutic applications as described elsewhere herein, e.g., in the general description for physiological or developmental abnormalities, or below in the description of kits for diagnosis. See, e.g., Berkow (ed.) The Merck Manual of Diagnosis and Therapy, Merck & Co., Rahway, N.J.; Thorn, et al. Harrison's Principles of Internal Medicine McGraw-Hill, NY; Gilman, et al. (eds. 1990) Goodman and Gilman's: The Pharmacological Bases of Therapeutics 8th Ed., Pergamon Press; (1990) Remington's Pharmaceutical Sciences (18th ed.) Mack Publishing Co., Easton, Pa.; Langer (1990) Science 249:1527-1533; Merck Index, Merck & Co., Rahway, N.J.; Avis, et al. (eds. 1993) Pharmaceutical Dosage Forms: Parenteral Medications 2d ed., Dekker, NY; Lieberman, et al. (eds. 1990) Pharmaceutical Dosage Forms: Tablets 2d ed., Dekker, NY; Lieberman, et al. (eds. 1990) Pharmaceutical Dosage Forms: Disperse Systems Dekker, NY; Fodor, et al. (1991) Science 251:767-773, Coligan Current Protocols in Immunology; Hood, et al. Immunology Benjamin/Cummings; Paul (ed.) Fundamental Immunology; Methods in Enzymology Academic Press; Parce, et al. (1989) Science 246:243-247; Owicki, et al. (1990) Proc. Nat'l Acad. Sci. USA 87:4007-4011; and Blundell and Johnson (1976) Protein Crystallography, Academic Press, New York; each of which is incorporated herein by reference. Additional uses will be apparent to a person having ordinary skill in the art in light of the teachings provided herein.

IX. Kits

This invention also contemplates use of ligand OX2 proteins, fragments thereof, peptides, and their fusion products and related reagents will also be useful in a variety of diagnostic kits and methods for detecting the presence of a binding composition as described in, e.g., Harlow and Lane (1988) Antibodies: A Laboratory Manual CSH; U.S. Pat. No. 3,645,090; U.S. Pat. No. 3,940,475; Rattle, et al. (1984) Clin. Chem. 30:1457-1461; U.S. Pat. No. 4,659,678; and Viallet, et al. (1989) Progress in Growth Factor Res. 1:89-97; each of which is incorporated herein by reference.

The broad scope of this invention is best understood with reference to the following examples, which are not intended to limit the invention to specific embodiments.

### EXAMPLES

I. General Methods

Some of the standard methods are described or referenced, e.g., in Maniatis, et al. (1982) Molecular Cloning: A Laboratory Manual Cold Spring Harbor Laboratory, Cold Spring Harbor Press; Sambrook, et al. (1989) Molecular Cloning: A Laboratory Manual (2d ed.) vols. 1-3, CSH Press, NY; Ausubel, et al. (1987 and Supplements) Current Protocols in Molecular Biology, Greene/Wiley, New York; or Innis, et al. (eds. 1990) PCR Protocols: A Guide to Methods and Applications Academic Press, N.Y. Methods for protein purification include such methods as ammonium sulfate precipitation, column chromatography, electrophoresis, centrifugation, crystallization, and others. See, e.g., Ausubel, et al. (1987 and periodic supplements); Coligan, et al. (eds. 1995 and periodic supplements) Current Protocols in Protein Science Wiley & Sons; Deutscher (1990) “Guide to Protein Purification” in Methods in Enzymology, vol. 182, and other volumes in this series; and manufacturer's literature on use of protein purification products, e.g., Pharmacia, Piscataway, N.J., or Bio-Rad, Richmond, Calif. Combination with recombinant techniques allow fusion to appropriate segments, e.g., to a FLAG sequence or an equivalent which can be fused via a protease-removable sequence. See, e.g., Hochuli (1990) “Purification of Recombinant Proteins with Metal Chelate Absorbent” in Setlow (ed.) Genetic Engineering, Principle and Methods 12:87-98, Plenum Press, N.Y.; and Crowe, et al. (1992) QIAexpress: The High Level Expression & Protein Purification System QUIAGEN, Inc., Chatsworth, Calif.

FACS analyses are described in Melamed, et al. (1990) Flow Cytometry and Sorting Wiley-Liss, Inc., New York, N.Y.; Shapiro (1988) Practical Flow Cytometry Liss, New York, N.Y.; and Robinson, et al. (1993) Handbook of Flow Cytometry Methods Wiley-Liss, New York, N.Y.

II. Isolation of a DNA Clone Encoding Ligand OX2 Protein

Isolation of human ligand OX2 is described in McCaughan, et al. (1987) Immunogenetics 25:329-335. Standard hybridization methods can be used, or PCR primers constructed to isolate the clone. Entrez accession numbers for both the nucleotide and amino acid sequences are provided above.

Various cells are screened using an appropriate probe for high level message expression, and expression distribution has been published. Appropriate cells are selected as sources for cDNA cloning, e.g., using standard methods of PCR or hybridization.

Standard PCR techniques are used to amplify an OX2 gene sequence from genomic DNA or an OX2 or fragment from cDNA derived from mRNA. Appropriate primers are selected from the sequences described, and a full length clone is isolated. Various combinations of primers, of various lengths and possibly with differences in sequence, may be prepared. The full length clone can be used as a hybridization probe to screen for other homologous genes using stringent or less stringent hybridization conditions.

In another method, oligonucleotides are used to screen a library. In combination with polymerase chain reaction (PCR) techniques, synthetic oligonucleotides in appropriate orientations are used as primers to select correct clones from a library.

III. Large Scale Production of OX2

For in vitro or in vivo biological assays, OX2 or OX2-E-tag are produced, e.g., in large amounts with transfected COS-7 cells grown in RPMI medium supplemented with 1% Nutridoma HU (Boehringer Mannheim, Mannheim, Germany) and subsequently purified. Adenovirus expression systems may be used.

Recombinant protein may be purified using standard procedures. Affinity chromatography of epitope tagged fusion protein may be utilized.

IV. Preparation of Antibodies Specific for OX2

Inbred Balb/c mice are immunized, e.g., with 1 ml of purified OX2 emulsified in Freund's complete adjuvant on day 0, and in Freund's incomplete adjuvant on days 15 and 22. The mice are boosted with 0.5 ml of purified OX2 administered intravenously.

Hybridomas are created, e.g., using the non-secreting myeloma cells line SP2/0-Ag8 and polyethylene glycol 1000 (Sigma, St. Louis, Mo.) as the fusing agent. Hybridoma cells are placed in a 96-well Falcon tissue culture plate (Becton Dickinson, NJ) and fed with DMEM F12 (Gibco, Gaithersburg, Md.) supplemented with 80 ug/ml gentamycin, 2 mM glutamine, 10% horse serum (Gibco, Gaithersburg, Md.), 1% ADCM (CRTS, Lyon, France) 10⁻⁵ M azaserine (Sigma, St. Louis, Mo.) and 5×10⁻⁵ M hypoxanthine. Hybridoma supernatants are screened for antibody production against OX2, e.g., by immunocytochemistry (ICC) using acetone fixed OX2 transfected COS-7 cells and/or by ELISA using OX2 purified from COS-7 supernatants as a coating antigen. Aliquots of positive cell clones are expanded for 6 days and cryopreserved as well as propagated in ascites from pristane (2,6,10,14-tetramethylpentadecane, Sigma, St. Louis, Mo.) treated Balb/c mice who had received on intraperitoneal injection of pristane 15 days before. About 10⁵ hybridoma cells in 1 ml of PBS are given intraperitoneally, and 10 days later, ascites are collected from each mouse.

After centrifugation of the ascites, the antibody fraction may be isolated by ammonium sulfate precipitation and anion-exchange chromatography on a Zephyr-D silicium column (IBF Sepracor) equilibrated with 20 mM Tris pH 8.0. Proteins are eluted with a NaCl gradient (ranging from 0 to 1 M NaCl). 2 ml fractions may be collected and tested by ELISA for the presence of anti-OX2 antibody. The fractions containing specific anti-OX2 activity are pooled, dialyzed, and frozen.

V. Preparation of an OX2 Deletion Mouse.

OX2 knockout (KO) mice were made essentially according to the procedure described by Galli-Taliadoros, et al. (1995) J. Immunol. Methods 181:1-15; Korner, et al. (1997) Eur. J. Immunol. 27:2600-2609; and Lemckert, et al. (1997) Nucl. Acids Res. 25:917-918. In short, a C57BL/6 genomic library was screened using a PCR fragment of the mouse OX2 cDNA as a probe. The isolated genomic clone contained an insert of about 16 kB from which a 9.5 kB SalI fragment was sub-cloned into pBluescript. This clone contained part of intron I, exon II (encoding the signal peptide), intron II, exon III (encoding the V-like Ig domain), intron III, exon IV (encoding the C2-like Ig domain), and part of intron IV. From this clone a targeting construct was created by replacing an NcoI fragment encoding the C-terminal part of the V-like Ig domain with the Neomycin cassette and shortening the upstream part of the clone so that it contained only the 3′ part of the exon encoding the signal peptide. An ES cell line derived from C57BL/6J mice (Bruce 4; see Galli-Taliadoros, et al. (1995) J. Immunol. Methods 181:1-15 and Lemckert, et al. (1997) Nucl. Acids Res. 25:917-918) was transfected by electroporation, and G418 resistant colonies were isolated and screened for homologous recombination by PCR and Southern blotting. One homologous recombinant out of 1,000 clones was isolated and used to create chimeric mice. See Lemckert, et al. (1997) Nucl. Acids Res. 25:917-918. Male chimeras were bred with female wild type C57BL/6J mice and the offspring with black coat-color (indicating germ-line transmission) were screened for the presence of the targeted allele. Heterozygous F1 mice were inter-crossed to obtain homozygous knockout mice, which were used to establish a pure C57BL/6.OX2−/− breeding colony. Age a sex-matched wild type C57BL/6J mice were used as controls in all studies.

VI. Initial Observations on OX2−/− (Knockout) Mice

Analysis of OX2 KO vs. wild type (wt) mice involved a gross analysis of organ structures. At the macroscopic level, organ structures appeared normal, with the exception of mesenteric lymph nodes (MLN) that appeared “fused” together into one long tube-like structure. In wt mice, the normal MLN structure is characterized by separate lymph nodes joined by lymphatic vessels in a ‘string of pearls’ configuration. The spleen was slightly enlarged, as were some lymph nodes. Differences were more apparent at the histological level upon staining for a variety of leukocyte antigens. In particular, the red pulp of the spleen of OX2 KO mice appeared enlarged (but not edematous) and filled with F4/80+ cells, i.e., macrophages as it should be. The subpopulation of metallophilic macrophages surrounding the B cell follicles in spleen (MOMA-1+) were also increased by 2-3 fold. Gr-1+ cells, e.g., granulocytes, were also more numerous in OX2 KO mouse spleen, by a factor of about 2 fold. White pulp areas were of normal size. Thus, there appeared to be a relative expansion of myeloid lineage cells, including macrophages, in spleen which could possibly account for the increased size. The MLN “tube” consisted of clearly demarcated individual lymph node structures, but each attached together (fused) with what appeared to be an expanded paracortical or subcapsular region and this was positive also for F4/80+/MOMA-1+ macrophages. Cells appeared enlarged and activated and were MHC class II+.

Sections of CNS from wild-type (wt) and OX2 KO mice, as stained for microglia, the resident CNS macrophage, with an antibody to Mac-1 (CD11b). The major findings were:

(i) In spinal cord there appeared in OX2 KO mice to be an increase in numbers of microglia by around 20% relative to wt mice.

(ii) Small foci of microglial cells, occasionally even a structure resembling the microglial clustering associated with neuritic amyloid plaques, were observed in the OX2 KO spinal cord. Such foci are never seen in the normal healthy wt CNS, and indicates microglial cell activation, proliferation or clustering. Levels of CD45 expression were generally enhanced on microglia in OX2 KO mice. CD45 is usually low in normal microglia, but enhanced upon activation. See, e.g., Sedgwick, et al. (1991) Proc. Nat'l Acad. Sci. USA 88:7438-7442; Sedgwick, et al. (1993) J. Exp. Med. 177:1145-1152; Ford, et al. (1995) J. Imunol. 154:4309-4321; Ford, et al. (1996) J. Exp. Med. 184:1737-1745; Sedgwick and Hickey (1997) in Keane and Hickey (eds.) Immunology of the Nervous System Oxford Press, New York; and Sedgwick, et al. (1998) J. Immunol. 160:5320-5330.

These findings were consistent with the view that the loss of OX2 (in this case on neurons) leads to some degree of dysregulation of resident macrophages (being resident microglial cells in the CNS).

The general message from our studies in the OX2 KO mouse was that loss of this molecule released myeloid cells generally, and macrophage-lineage cells specifically, from normal regulation even in the steady state. It was possible, therefore, that in situations where macrophage activation and proliferation was enhanced (e.g., in pathological states) loss of OX2 may lead to an even greater or more rapid increase in macrophage activation.

To test this hypothesis, models of macrophage activation were chosen. The first was a facial nerve transection model to investigate CNS macrophage (microglial cell) activation within the downstream facial nucleus which follows cutting of the facial nerve. See Streit and Graeber (1993) Glia 7:68-74. This model is appropriate in the present case as it is known that it is the damaging effects of nerve transection on neurons (which are OX2-positive) that leads subsequently to a response by microglial cells (which are OX2-R-positive) within the facial nucleus. This response can be examined by immunohistological assessment of the facial nucleus.

According to this hypothesis, it is predicted that in the absence of OX2 on neurons in OX2 KO mice, the microglial cell response would be more rapid and of greater magnitude. Indeed this was found, particularly that two days after transection, microglial cell activation was already evident in OX2 KO but not wt mice. Moreover, the differences at day 4 between wt and OX2 KO mice were more apparent. By day 7, microglial cell activation was equivalent in both types of mice.

This experiment provides direct evidence that OX2 signals from a non-macrophage-lineage cell (in this case, the neuron) participate in macrophage regulation.

In a second model, mice were injected parenterally with lipopolysaccharide (LPS) known to induce rapid macrophage activation. Within 90 minutes, quantitation of serum TNF production is useful as a measure of macrophage activation. The OX2 KO mice should respond to much lower doses of LPS and with increased TNF production. This correlation has been confirmed; in certain cases, TNF production in these mice was 2-4 fold higher than in wild type mice. OX2 KO mice show an earlier and more accelerated onset of EAE relative to wt mice. The disease ultimately is not greater than wt mice, so, analogous to the microglia, the onset is fast but it ultimately does not exceed that in the wt control. Removal of the OX2 interaction with its receptor enhances the macrophage response, leading to greater or more rapid disease onset. The opposite effect would typically be desired therapeutically.

Thus, when macrophages are stimulated within the CNS or outside it, an OX2 negative environment leads to enhanced macrophage activity and function.

All references cited herein are incorporated herein by reference to the same extent as if each individual publication or patent application was specifically and individually indicated to be incorporated herein by reference.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

What is claimed is:

1. A method of modulating the trafficking or activation of a leukocyte in an animal, said method comprising contacting myeloid lineage cells in said animal with a therapeutic amount of:
a) an agonist of a mammalian OX2 protein; or
b) an antagonist of a mammalian OX2 protein.

2. The method of claim 1, wherein said:
a) mammalian OX2 protein is a primate protein;
b) antagonist is an antibody which binds to said mammalian OX2; or
c) said cells are monocyte/macrophage lineage cells.

3. The method of claim 2, wherein said myeloid lineage cells include a monocyte, macrophage, microglial, or dendritic cell.

4. The method of claim 1, wherein said animal exhibits signs or symptoms of an inflammatory, infective, leukoproliferative, neurodegenerative, or post-traumatic condition.

5. The method of claim 4, wherein said sign or symptom is in neural tissue; lymphoid tissue; myeloid tissue; pancreas; gastrointestinal tissue; thyroid tissue; muscle tissue; or skin or collagenous tissue.

6. The method of claim 1, wherein said modulating is inhibiting function of said leukocyte cell.

7. The method of claim 6, wherein said administering is said agonist.

8. The method of claim 7, wherein said agonist is said mammalian OX2.

9. The method of claim 7, wherein said animal is experiencing signs or symptoms of autoimmunity; an inflammatory condition; an infection; tissue specific autoimmunity; degenerative autoimmunity; rheumatoid arthritis; atherosclerosis; multiple sclerosis; vasculitides; delayed hypersensitivities; skin grafting; a transplant; spinal injury; stroke; neurodegeneration; or ischemia.

10. The method of claim 7, wherein said administering is in combination with:
a) an anti-inflammatory cytokine agonist or antagonist;
b) an analgesic;
c) an anti-inflammatory agent; or
d) a steroid.

11. The method of claim 1, wherein said modulating is enhancing function of said leukocyte cell.

12. The method of claim 11, wherein said administering is said antagonist.

13. The method of claim 12, wherein said antagonist is:
a) an antibody which binds to said mammalian OX2; or
b) a mutein of said mammalian OX2 which competes with said mammalian OX2 in binding to an OX2 receptor, but does not substantially signal.

14. The method of claim 12, wherein said animal experiences signs or symptoms of wound healing or clot formation.

15. The method of claim 12, wherein said administering is in combination with:
a) an angiogenic factor;
b) a growth factor, including FGF or PDGF;
c) an antibiotic; or
d) a clotting factor.

16. A method of modulating the activation of a leukocyte in a tissue, said method comprising contacting myeloid lineage cells in said tissue with:
a) an agonist of a mammalian OX2 protein; or
b) an antagonist of a mammalian OX2 protein.

17. The method of claim 16, wherein said modulating is inhibiting said leukocyte cell, and said contacting is with said agonist.

18. The method of claim 17, wherein said administering is in combination with:
a) an anti-inflammatory cytokine agonist or antagonist;
b) an analgesic;
c) an anti-inflammatory agent; or
d) a steroid.

19. The method of claim 16, wherein said modulating is enhancing, and said contacting is with said antagonist.

20. The method of claim 19, wherein said administering is in combination with:
a) an angiogenic factor;
b) a growth factor, including FGF or PDGF;
c) an antibiotic; or
d) a clotting factor.


SEQUENCE LISTING (3 sequences)

SEQ ID NO: 1 - protein, 274 aa, primate
VIRMPFSHLSTYSLVWVMAAVVLCTAQVQVVTQDEREQLYTTASLKCSLQNAQEALIVTWQKKKAVSPENMVTFSENHGVVIQPAYKDKINITQLGLQNSTITFWNITLEDEGCYMCLFNTFGFGKISGTACLTVYVQPIVSLHYKFSEDHLNITCSATARPAPMVFWKVPRSGIENSTVTLSHPNGTTSVTSILHIKDPKNQVGKEVICQVLHLGTVTDFKQTVNKGYWFSVPLLLSIVSLVILLVLISILLYWKRHRNQDRGELSQGVQKMT

SEQ ID NO: 2 - protein, 278 aa, rodent
MASLVFRRPFCHLSTYSLIWGMAAVALSTAQVEVVTQDERKALHTTASLRCSLKTSQEPLIVTWQKKKAVSPENMVTYSKTHGVVIQPAYKDRINVTELGLWNSSITFWNTTLEDEGCYMCLFNTFGSQKVSGTACLTLYVQPIVHLHYNYFEDHLNITCSATARPAPAISWKGTGTGIENSTESHFHSNGTTSVTSILRVKDPKTQVGKEVICQVLYLGNVIDYKQSLDKGFWFSVPLLLSIVSLVILLVLISILLYWKRHRNQERGESSQGMQRMK

SEQ ID NO: 3 - protein, 278 aa, rodent
MGSPVFRRPFCHLSTYSLLWAIAAVALSTAQVEVVTQDERKLLHTTASLRCSLKTTQEPLIVTWQKKKAVGPENMVTYSKAHGVVIQPTYKDRINITELGLLNTSITFWNTTLDDEGCYMCLFNMFGSGKVSGTACLTLYVQPIVHLHYNYFEDHLNITCSATARPAPAISWKGTGSGIENSTESHSHSNGTTSVTSILRVKDPKTQVGKEVICQVLYLGNVIDYKQSLDKGFWFSVPLLLSIVSLVILLVLISILLYWKRHRNQERGESSQGMQRMK